(19) United States
(12) Patent Application Publication
(10) Pub. No.: US 2010/0228239 A1
(43) Pub. Date: Sep. 9, 2010

(54) ABLATION DEVICE WITH SUCTION CAPABILITY

(75) Inventor: David Freed, Westborough, MA (US)

Correspondence Address: VISTA IP LAW GROUP LLP, 12930 Saratoga Avenue, Suite D-2, Saratoga, CA, 95070, US

(73) Assignee: CYTYC CORPORATION, Marlborough, MA (US)

(21) Appl. No.: 12/717,017

(22) Filed: Mar. 3, 2010

Related U.S. Application Data
(60) Provisional application No. 61/158,537, filed on Mar. 9, 2009.

(51) Int. Cl. A61B 18/04; A61B 18/14; A61M 25/10

(52) U.S. Cl. 606/27; 606/41; 604/103.01

(57) ABSTRACT

An ablation device includes an inflatable member having a cavity, a layer surrounding at least part of the inflatable member, wherein the layer includes one or more ablation elements for providing ablation energy, and a plurality of channels located between the layer and the inflatable member, wherein the layer has a plurality of openings that are in fluid communication with the plurality of channels. An ablation device includes an inflatable member having a cavity, and a layer surrounding at least part of the inflatable member, wherein the layer includes one or more ablation elements for providing ablation energy, wherein the layer has a plurality of openings, and is spaced away from at least a portion of the inflatable member.

### RELATED APPLICATION DATA

The present application claims the benefit under 35 U.S.C. §119 to U.S. provisional application Ser. No. 61/158,537 filed Mar. 9, 2009. The foregoing application is hereby incorporated by reference into the present application in its entirety.

### FIELD

The inventions disclosed in this application relate generally to systems and methods for treating internal body organ tissue, such as the endometrium of the uterus, with ablation energy.

### BACKGROUND

There are certain medical procedures that are carried out within a body cavity. One example of such a procedure is tissue ablation. Ablation of the interior lining of a body organ is a procedure which involves heating the organ lining to temperatures which destroy the cells of the lining or coagulate tissue proteins. Such a procedure may be performed as a treatment to one of many conditions, such as chronic bleeding of the endometrial layer of the uterus or abnormalities of the mucosal layer of the gallbladder. Existing methods for effecting ablation include circulation of heated fluid inside the organ (either directly or inside a balloon), laser treatment of the organ lining, and resistive heating using application of RF energy to the tissue to be ablated.

Sometimes, the ablation process creates steam as tissue is being ablated. For example, in one procedure in which RF energy is used to treat tissue at the uterine wall, steam is created as the tissue is being ablated. The steam may escape from the uterine wall and travel to the fallopian tubes or other parts in the body. Since the steam carries some energy, it may damage healthy tissue. The steam may also exit from the vaginal opening into the area outside the patient in the operation room. This is unsightly, and in some cases, may block the view of a surgeon.

### SUMMARY

In accordance with some embodiments, an ablation device includes an inflatable member having a cavity, a layer surrounding at least part of the inflatable member, wherein the layer includes one or more ablation elements for providing ablation energy, and a plurality of channels located between the layer and the inflatable member, wherein the layer has a plurality of openings that are in fluid communication with the plurality of channels.

In accordance with some embodiments, an ablation device includes an inflatable member having a cavity, and a layer surrounding at least part of the inflatable member, wherein the layer includes one or more ablation elements for providing ablation energy, wherein the layer has a plurality of openings, and is spaced away from at least a portion of the inflatable member.

In accordance with some embodiments, a method for use in an ablation process includes inflating a member inside a cavity to push an ablation element towards a tissue, and removing fluid inside cavity using a space that is between the ablation element and the inflated member.

Other and further aspects and features will be evident from reading the following detailed description of the embodiments, which are intended to illustrate, not limit, the invention.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates an ablation device having an inflatable member and an outer layer for delivering ablation energy in accordance with some embodiments.

FIG. 2 illustrates the ablation device of FIG. 1, particularly showing the inflatable member and the outer layer in a deployed state.

FIG. 3 illustrates part of the ablation of FIG. 1, particularly showing an end view of a sheath that is used to carry the inflatable member in accordance with some embodiments.

FIG. 4 illustrates part of the ablation device of FIG. 1, particularly showing a plurality of channels.

FIG. 5 illustrates a cross sectional view of the distal end of the ablation device of FIG. 1 in accordance with some embodiments.

FIGS. 6A-6D illustrate a method of using the ablation device of FIG. 1.

FIG. 7 illustrates an ablation device in accordance with other embodiments.

FIG. 8 illustrates an ablation device in accordance with other embodiments.

FIG. 9 illustrates an ablation device in accordance with other embodiments.

### DESCRIPTION OF THE EMBODIMENTS

Various embodiments are described hereinafter with reference to the figures. It should be noted that the figures are not drawn to scale and that elements of similar structures and/or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment, but instead may be practiced in many other embodiments, even if not so illustrated.

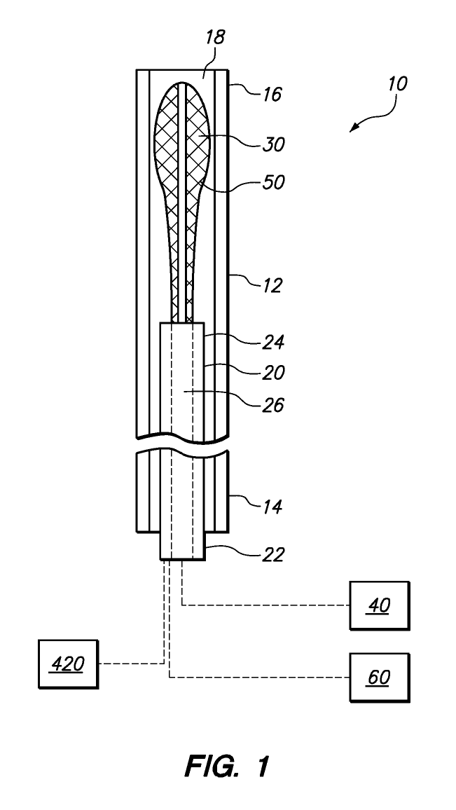

FIG. 1 illustrates an ablation device 10 in accordance with some embodiments. The ablation device 10 includes an outer sheath 12 having a proximal end 14, a distal end 16, and a lumen 18 extending between the ends 14, 16. The ablation device 10 also includes an inner sheath 20 that is slidably disposed within the lumen 18 of the outer sheath 12, and includes a proximal end 22, a distal end 24, and a lumen 26 extending between the ends 22, 24.

An inflatable member 30 is coupled to the distal end 24 of the inner sheath 20, and a fluid source 40 is coupled to the proximal end 22 of the inner sheath 20. The fluid source 40 is for delivering fluid for inflating the inflatable member 30. The fluid may be any liquid, such as saline, or gas. As shown in the figure, the ablation device also includes an outer layer 50 surrounding the inflatable member 30, wherein the outer layer 50 is for delivering ablation energy to treat tissue. In the illustrated embodiments, the ablation device 10 includes a radio-frequency (RF) generator 60 for providing energy to the outer layer 50.

As shown in the figure, the inflatable member 30 is in an un-inflated state and is housed within the lumen 18 of the outer sheath 12. Also, the layer 50 is in an un-deployed configuration when it is housed within the lumen 18 of the outer sheath 12. During use, the inflatable member 30 is deployed out of the lumen 18 by advancing the inner sheath 20 distally relatively to the outer sheath 12. After the inflatable member 30 is deployed, the inflation fluid is then delivered from the fluid source 40 to inflate the member 30. Inflation of the member 30 causes the outer layer 50 to expand (e.g., by flexing and/or stretching) to a deployed configuration. Alternatively, inflation of the member 30 can be more of an unfolding event, where the surface area remains relatively constant, but the internal volume expands.

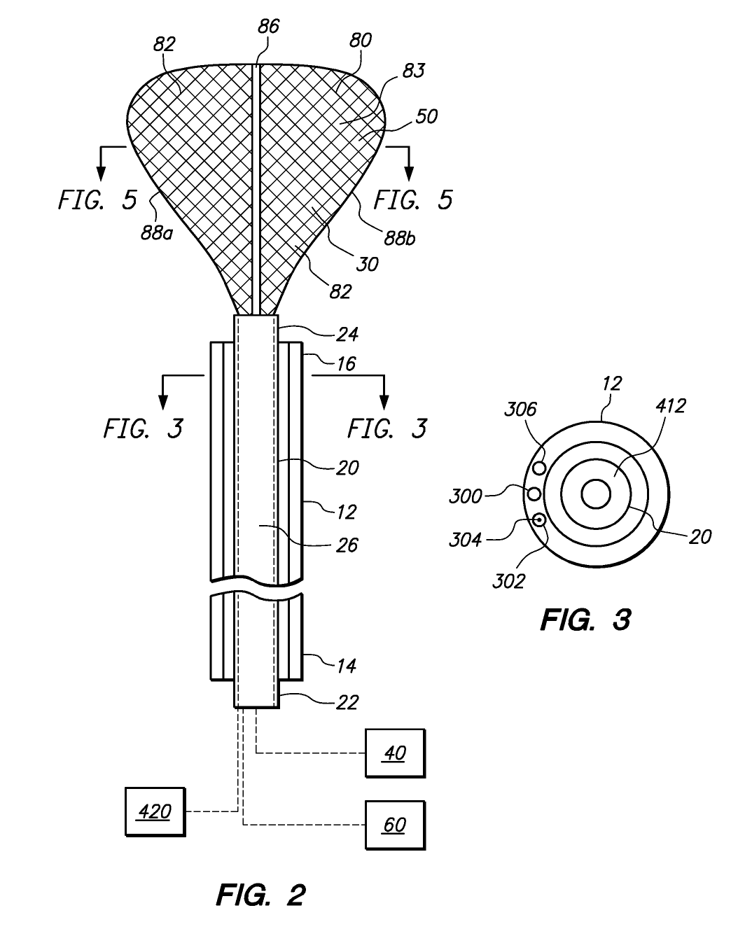

FIG. 2 illustrates the ablation device 10 of FIG. 1, showing the inflatable member 30 deployed out of the lumen of the outer sheath 12, and has been inflated by fluid delivered from the fluid source 40. The inflated member 30 correspondingly causes the layer 50 to be deployed to its deployed configuration. In its deployed configuration, the shape of the ablation device 10 preferably conforms to the internal shape of the organ being treated.

In some embodiments, the outer sheath 12 is provided with a light source 300, a first lumen 302 for allowing an endoscope 304 to be placed therein, and an (optional) second lumen 306 for allowing another device, such as a biopsy device, an aspiration device, a clamp, a guidewire, etc., to be placed therein (FIG. 3). The proximal end of the endoscope 304 may be coupled to a screen for allowing a physician to see the inside of the patient during a procedure.

The inflatable member 30 may be made from any material, such as a polymer. In some embodiments, the inflatable member 30 has a shape (un-inflated shape and/or inflated shape) that resembles a shape of a uterine cavity. As shown in FIG. 2, the inflated shape (in the elevation view) of the member 30 resembles a shape of the uterine cavity. In other embodiments, the inflated shape of the member 30 in the end view may also resemble the shape of the uterine cavity. In other embodiments, the inflatable member 30 may have other shapes. Also, in some embodiments, the member 30 is made from a compliant material, which allows the member 30 to conform to the shape of the cavity when the member 30 has been inflated inside the cavity.

In the illustrated embodiments, the outer layer 50 is an array structure 80 that surrounds at least parts of the inflatable member 30. The array structure 80 includes a plurality of conductive elements such as metallic traces or wires 82, and a plurality of openings 83 for allowing fluid, such as steam, to flow therethrough. One or more of the conductive elements 82 may function as active electrode(s), and another one or more of the conductive elements 82 may function as return electrode(s).

The active and return electrodes are electrically coupled to the RF generator 60 via conductor leads (not shown), which provides power to cause a current to be generated between the active and return electrodes. The conductor leads may be housed in a lumen within a wall of the inner sheath 20. Alternatively, the conductor leads may be housed within the lumen 26 of the inner sheath 20. In the illustrated embodiments, the conductive elements 82 are oriented at a slanted angle relative to the longitudinal axis of the inner sheath 20. In other embodiments, the conductive elements 82 may be oriented at different angles. For example, in other embodiments, a first set of the wires 82 may be parallel to the longitudinal axis of the inner sheath 20, and a second set of the wires 82 may be perpendicular to the first set of wires 82. Alternatively, the wires may be knitted into an omni-directional surface.

The active electrode(s) may be electrically insulated from the return electrode(s) by providing an insulator that separates the active electrode(s) from the return electrode(s). In the illustrated embodiments, the insulator 86 divide the array structure 80 into two electrode regions 88a, 88b. In other embodiments, the ablation device 10 may include more than one insulator 86. For example, in other embodiments, the ablation device 10 may include an additional insulator that is perpendicular to that shown in the figure, thereby dividing the array structure 80 into four electrode regions, with two regions being active electrodes and the other two regions being return electrodes. In further embodiments, the array structure 80 may be divided by insulated regions into a variety of other electrode configurations. The insulating regions may be formed by etching or other techniques to divide the array structure 80 into electrode regions.

In other embodiments, the active electrode(s) may be electrically insulated from the return electrode(s) by knitting the array structure 80 and insulator 86 in a certain pattern.

In other embodiments, the array structure 80 may be constructed using material that is electrically non-conductive. In such cases, one or more active electrode(s) and one or more return electrode(s) may be secured to the array structure 80.

In some embodiments, the array structure 80 is formed of a stretchable metalized fabric mesh, which may be knitted from a nylon and spandex knit plated with gold or other conductive material. In one array design, the knit is formed of three monofilaments of nylon knitted together with single yarns of spandex. Each yarn of spandex has a double helix of five nylon monofilaments coiled around it.

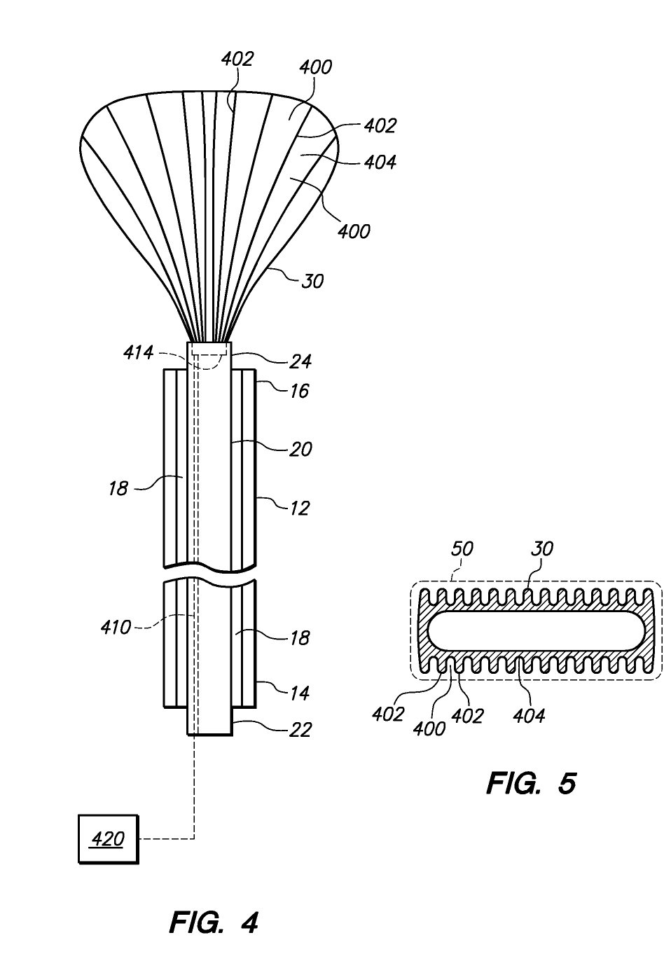

The ablation device 10 also includes a plurality of channels 400 that are between the layer 50 and a surface of the inflatable member 30 (FIGS. 4 and 5). FIG. 4 illustrates the ablation device 10 of FIG. 1, but with the layer 50 removed to show the channels 400. In the illustrated embodiments, each channel 400 is formed by providing two ridges 402 that are spaced away from each other and are disposed on the surface 404 of the inflatable member 30. The ridges 402 may be formed integrally together with the inflatable member 30. Alternatively, the ridges 402 may be secured to the surface 404 of the inflatable member 30 using an adhesive. The ridges 402 also function to space the layer 50 away from the surface 404 of the inflatable member 30, thereby preventing the layer 50 from contacting the surface 404 to maintain the channels 400 open. In other embodiments, instead of having a plurality of channels 400, the ablation device 10 may include only one channel 400.

As shown in FIG. 4, the inner sheath 20 includes a lumen 410 within a wall 412 (FIG. 3) of the inner sheath 20. The lumen 410 is in fluid communication with a duct 414, which is configured to collect fluid from the channels 400. The lumen 410 is in fluid communication with a suction device (or vacuum source) 420 coupled to the proximal end 22 of the inner sheath 20. During use, the suction device 420 is activated to create a suction within the lumen 410, thereby drawing fluid from the channels 400 into the lumen 410 to remove fluid, such as steam, that may be generated from tissue ablation using the layer 50. In a presently preferred embodiment, instead of providing the lumen 410 at the wall 420 of the inner sheath 20, the lumen 410 is provided using a length of tubing housed within the lumen 26 of the inner sheath. In other embodiments, the ablation device 10 may have a plurality of lumens 410, each in fluid communication with one or more channels 400. In yet another embodiment, the duct 414 routes the fluid collected in the channel(s) 400 to the lumen 18 between inner sheath 20 and outer sheath 12.

Although the channel(s) 400 and the lumen(s) 410 of the ablation device 10 have been described with reference to removing fluid from an ablation site, in other embodiments, the channel(s) 400 and the lumen(s) 410 may be used to deliver fluid or object(s) to the target site. For example, in other embodiments, the channel(s) 400 and the lumen(s) 410 may deliver saline or a drug to the ablation site. In such cases, instead of the suction device 420, the ablation device 100 is coupled to a fluid delivery source that is configured to deliver the fluid (e.g., saline, drug, etc.) through the respective fluid “delivery” lumen(s) 410 and channels 400. Furthermore, the ablation device 100 may be alternately coupled between a fluid delivery device and a fluid aspiration device, wherein such devices use respective dedicated fluid delivery and aspiration lumens 410 in fluid communication with one or more of the channels 400, or wherein such devices share a common (i.e., combined function) fluid delivery and aspiration lumen in fluid communication with one or more channels 410.

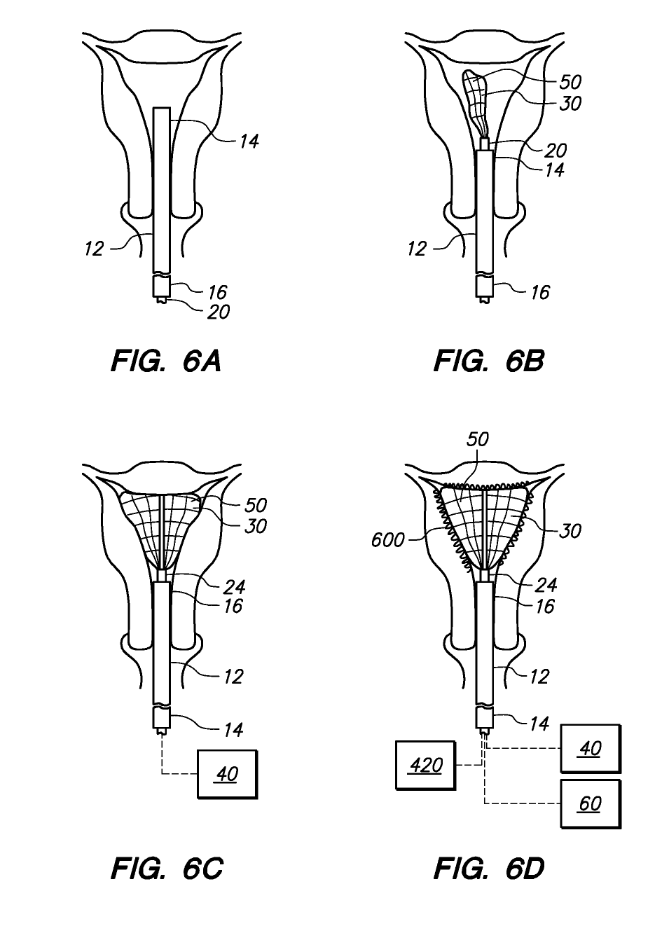

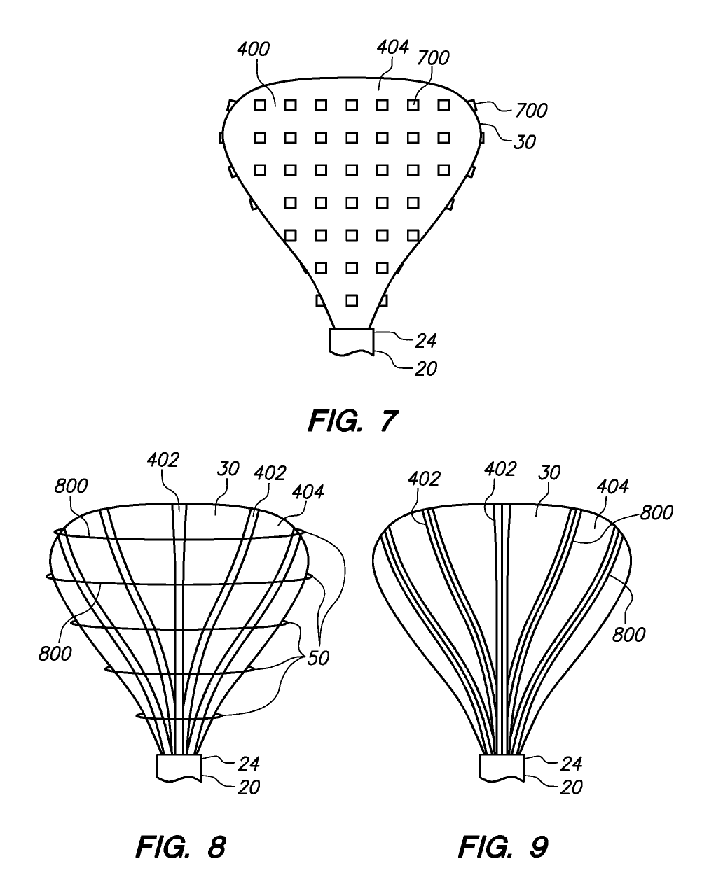

FIGS. 6A-6D illustrate a method of using the ablation device 10 in accordance with some embodiments. The method will be discussed with reference to treating tissue at a uterine wall. However, it should be understood that the ablation device 10 may be used to treat tissue at other locations in the patient's body in other embodiments.

First, before the ablation is performed, an optional assessment procedure is performed for detecting perforation in the uterine cavity. For such purpose, a fluid (either liquid or gas) is delivered into the uterine cavity to slightly pressurize the cavity. A pressure sensing system monitors the pressure within the cavity for a predetermined test period. If cavity pressure is not substantially sustained during the test period, the physician is alerted to further assess the cavity for perforations before initiating treatment within the uterine cavity.

To perform the ablation procedure, the outer sheath 12 is first inserted into the uterus, with the outer sheath 12 covering the inflatable member 30 to compress the inflatable member 30 into a confined configuration (FIG. 6A). Once the distal end 14 of the outer sheath 12 is within the uterus, the outer sheath 12 is retracted proximally relative to the inner sheath 20, thereby deploying the inflatable member 30 out of the lumen 18 of the outer sheath 12 (FIG. 6B). Alternately, deployment of the inflatable member 30 can be initiated by its inflation.

The fluid source 40 then delivers inflation fluid to inflate the inflatable member 30 (FIG. 6C). The fluid may be delivered using the lumen 26 of the inner sheath 20. Alternatively, a separate tube may be provided within the inner sheath 20 for delivering the inflation fluid. In the illustrated embodiments, the inflatable member 30 is inflated until the layer 50 is in contact with tissue at the uterine wall. In some cases, the inflatable member 30 may be further inflated to press the layer 50 against the tissue.

The RF generator 60 is then used to supply ablation power to electrodes at the layer 50, thereby ablating tissue 600 at the uterine wall (FIG. 6D). The tissue at the uterine wall is heated as the RF energy passes from electrodes to the tissue, causing fluid (e.g., steam, moisture) to be released from the tissue. While the tissue is being ablated, the suction device 420 is activated to draw the fluid from the uterine cavity through openings at the layer 50 and into the channel(s) 400, which delivers the fluid to the proximal end (e.g., to a collection reservoir) via the lumen(s) 410. This fluid removal feature of the ablation device 10 is advantageous in that removing fluid, e.g., steam, from the ablation site minimizes the amount of uncontrolled thermal ablation that would otherwise be caused by the steam. Greater control over ablation depth is thus achieved by allowing ablation to occur only (or primarily) by RF energy rather than by thermal conduction. Also, as can be appreciated, spacing the layer 50 from the member 30 is advantageous in that it provides a space for allowing fluid (such as steam) that may be created during the ablation process to be collected, while at the same time allowing the member 30 to push the layer 50 towards the tissue for maintaining contact between the layer 50 and the tissue during the ablation process.

In some embodiments, during the operation of the ablation device 10, a sealing device (not shown) may provided to help prevent fluid from leaking out of the uterus. In some cases, the sealing device may also assist the suction device 420 in creating suction within the uterus during use.

After the tissue at the uterine wall has been desirably ablated, the RF generator 60 and the suction device 420 are deactivated. The inflated member 30 is then deflated by withdrawing fluid inside the member 30. After the member 30 has been deflated, the outer sheath 12 is then advanced distally relative to the inner sheath 20 to bring the member 30 into the lumen 18 of the outer sheath 12. Alternatively, the inner sheath 20 may be retracted proximally relative to the outer sheath 12 to bring the member 30 into the lumen 18 of the outer sheath 12. The outer sheath 12 carrying the inner sheath 20 and the member 30 is then removed from the patient. In another embodiment, the member 30 is deflated and withdrawn from the patient without being withdrawn in to inner sheath 20.

In the above embodiments, the ablation device 10 is described as having ridges 402 that form a channel 400. In other embodiments, instead of having elongate protrusions that define the channel 400, the ablation device 10 may include a plurality of block-like structures (or protrusions) 700 that define the channel(s) 400. In the figure, the layer 50 is not shown to show the inflatable member 30. Thus, as used in this specification, the term “channel” is not limited to a fluid delivery conduit defined by continuous structures, and may refer to any space through which a fluid may flow or travel. The structures 700 may be formed integrally together with the inflatable member 30. Alternatively, the structures 700 may be secured to the surface 404 of the inflatable member 30 using an adhesive. The structures 700 also function to space the layer 50 away from the surface 404 of the inflatable member 30, thereby preventing the layer 50 from contacting the surface 404 to maintain the channels 400 open. In other embodiments, instead of having a block-like configuration, each structure 700 may have other shapes and sizes. For example, in other embodiments, the structure 700 may have a grid configuration.

Also, in other embodiments, instead of a knitted structure, the layer 50 may include a plurality of wires that are not knitted together. FIG. 8 illustrates a variation of the layer 50 in accordance with some embodiments. The layer 50 includes a plurality of wires 800, wherein one or more of the wires function as active electrode(s), and another one or more of the wires function as return electrode(s). The array of wires 800 is supported on, and spaced away from the surface 404 of the member 300 by, elongated protrusions (e.g., ridges) 402 that are disposed on the surface 404 of the member 300. In the illustrated embodiments, the wires 800 are oriented such that they form an angle (e.g., an approximately 90° angle) with the elongated protrusions 402. In other embodiments, the wires 800 and the elongated protrusions 402 may align with each other (FIG. 9). Also, in other embodiments, the wires 800 may be supported on structures having other configurations, such as block-like configurations, or a grid-like configuration.

It should be noted that the inflatable member 30 and the array 50 described herein may be incorporated in other devices in other embodiments. For example, in other embodiments, the inflatable member 30 and the array 50 may be used with the ablation device described in U.S. Pat. No. 5,769,800, the entire disclosure of which is expressly incorporated by reference.

In other embodiments, instead of using RF energy for ablating tissue, the ablation device 10 may use other types of energy. For example, in other embodiments, the layer 50 may include one or more transducers for delivering ultrasound energy, one or more heat delivery devices (such as a device for delivering heated fluid, or a heating element) for delivering heat, or other types of devices that can be used to treat tissue.

Although particular embodiments have been shown and described, it will be understood that they are not intended to limit the disclosed inventions, and it will be obvious to those skilled in the art that various changes and modifications may be made without departing from the scope of the disclosed inventions. The specification and drawings are, accordingly, to be regarded in an illustrative rather than restrictive sense, and the disclosed inventions are intended to cover alternatives, modifications, and equivalents of the illustrated embodiments, as defined and limited only by the following claims. By way of non-limiting example, in an alternative embodiment, the no outer sheath 12 is provided, and thus there is no relative motion between outer sheath 12 and inner sheath 20.

What is claimed is:

1. An ablation device configured for ablating tissue within an internal body cavity, comprising:
an inflatable member defining an interior cavity;
an outer layer surrounding at least a portion of the inflatable member, the layer including one or more operative elements for providing ablation energy to tissue, wherein an exterior surface of the inflatable member and an inner surface of the outer layer define a plurality of channels disposed between the outer layer and the inflatable member, and wherein the outer layer has a plurality of openings in fluid communication with the respective plurality of channels; and
a fluid delivery and/or aspiration lumen in fluid communication with at least one of the plurality of channels.

2. The ablation device of claim 1, wherein the inflatable member, when inflated, has a shape of a uterine cavity.

3. The ablation device of claim 1, wherein the inflatable member has a plurality of protrusions formed on an outer surface thereof, including first and second protrusions spaced apart to form at least a portion of one of the plurality of channels.

4. The ablation device of claim 3, wherein the first and second protrusions comprise respective first and second ridges.

5. The ablation device of claim 3, wherein the first and second protrusions each have a block configuration.

6. The ablation device of claim 1, wherein the fluid delivery and/or aspiration lumen comprises an aspiration lumen, the ablation device further comprising a suction device in fluid communication with the aspiration lumen, the suction device configured to draw steam vapor through the respective openings and channels and into the aspiration lumen.

7. The ablation device of claim 1, the outer layer comprising an array of conductive wires configured such that, in use, a first part of the array provides a positive polarity, and a second part of the array provides a negative polarity.

8. The ablation device of claim 1, wherein the openings are substantially uniformly spaced apart in the outer layer.

9. The ablation device of claim 1, wherein the fluid delivery and/or aspiration lumen comprises a fluid delivery lumen, the ablation device further comprising a fluid source in fluid communication with the fluid delivery lumen.

10. An ablation device configured for ablating tissue within an internal body cavity, comprising:
an inflatable member defining an interior cavity; and
an outer layer surrounding at least a portion of the inflatable member, the layer including one or more operative elements for providing ablation energy to tissue, wherein the outer layer has a plurality of openings therethrough, and is spaced away from at least a portion of the inflatable member,
wherein the inflatable member has a plurality of spaced apart protrusions formed on an outer surface thereof, the spaced apart protrusions defining a plurality of channels in fluid communication with the respective plurality of openings through the outer layer.

11. The ablation device of claim 10, wherein the inflatable member, when inflated, has a shape of a uterine cavity.

12. The ablation device of claim 10, further comprising a suction device in fluid communication with one or more of the plurality of channels via an aspiration lumen extending through the ablation device, the suction device configured to draw steam vapor through one or more of the respective openings in the outer layer, and through one or more of the channels, and into the aspiration lumen.

13. The ablation device of claim 10, wherein the outer layer comprises an array of conductive wires configured such that, in use, a first part of the array provides a positive polarity, and a second part of the array provides a negative polarity.

14. The ablation device of claim 10, wherein the openings are substantially uniformly spaced apart in the outer layer.

15. The ablation device of claim 10, further comprising a fluid source in fluid communication with one or more of the plurality of channels via a fluid delivery lumen extending through the ablation device, so as to allow infusion of fluid from the fluid source through the respective fluid delivery lumen and one or more channels, and through the respective openings in the outer layer.

16. A method for ablating the wall tissue of an internal body cavity, comprising:
inflating a member inside the internal body cavity to thereby push an ablation element coupled to an exterior wall of the member into contact with the wall tissue;
using the ablation element to deliver energy to the wall tissue; and
removing fluid from inside the cavity through a channel extending between the ablation element and the inflated member.

17. The method of claim 16, wherein the ablation element comprises a wire electrode.

18. The method of claim 16, wherein the ablation element is carried by a bendable structure.

19. The method of claim 18, wherein the bendable structure comprises a knitted structure.

20. The method of claim 16, wherein the channel is formed by a plurality of ridges extending from an outer surface of the member.

21. The method of claim 16, wherein the channel is formed by a plurality of blocks located on an outer surface of the member.

22. A method for ablating the wall tissue of an internal body cavity, comprising:
inflating a member inside the internal body cavity to thereby push an ablation element coupled to an exterior wall of the member into contact with the wall tissue;
using the ablation element to deliver energy to the wall tissue; and
delivering fluid to the wall tissue, wherein the fluid passes through a fluid delivery lumen passing through the inflated member, through a channel formed between the ablation element and the inflated member in fluid communication with the fluid delivery lumen, and out an opening formed in the ablation element in fluid communication with the channel.

23. The method of claim 22, wherein the fluid comprises a therapeutic drug.
